(19) United States
(12) Patent Application Publication
(10) Pub. No.: US 2006/0069446 A1
(43) Pub. Date: Mar. 30, 2006

(54) Articular interposition implant

(76) Inventors: Mathieu Antoine, Joseph Ragusa, Meylan (FR); Patrice Francois Diebold, Nancy (FR)

Correspondence Address: THOMAS, KAYDEN, HORSTEMEYER & RISLEY, LLP, 100 GALLERIA PARKWAY, NW, STE 1750, ATLANTA, GA, 30339-5948, US

(21) Appl. No.: 11/232,044

(22) Filed: Sep. 21, 2005

(30) Foreign Application Priority Data
Sep. 21, 2004 (FR) FR-04 09955

(51) Int. Cl. A61F 2/42; A61F 2/30

(52) U.S. Cl. 623/21.11; 623/21.15; 623/18.11

(57) ABSTRACT

Articular interposition implant to be placed between the articular surfaces of at least two bones separated by an articular interface to achieve arthroplasty of a joint, said implant (1) comprising immobilization means shaped so that when the immobilization means are arranged in the articular interface, the immobilization means are supportive of at least one of the articular surfaces, wherein said implant (1) extends along a longitudinal axis (X-X′) and wherein said immobilization means are shaped to prevent rotation of said implant (1) on itself with respect to said longitudinal axis (X-X′).

### CROSS-REFERENCE TO RELATED APPLICATION

This Application claims priority to copending French Patent Application entitled, “Articular Interposition Implant” having Application No. FR-04 09955, filed on Sep. 21, 2004, which is entirely incorporated herein by reference.

### TECHNICAL FIELD

This invention relates to the technical field of articular interposition implants designed to be implanted, temporarily or permanently, in the articular interface between two bones, for example a metatarsal and a phalanx.

This invention more particularly relates to an articular interposition implant designed to be put in place between the articular surfaces of at least two bones separated by an articular interface, for the purpose of achieving arhroplasty of a joint. In this case, it is a matter of enabling the patient to recover the mobility of said joint and, together with this, eliminating or attenuating the pain connected with the degradation of the tissues and/or the inflammation of the articular area.

### BACKGROUND

There are several categories of articular prostheses routinely used in orthopaedic surgery and designed to replace the degraded articular surfaces.

The first category of prosthesis is generally formed from two implants each fixed, respectively, to one of the bones of the joint, the implants being set up mobile with respect to each other so as to enable the mobility of the joint. Such prostheses are generally permanent prostheses on account of their necessary fixation to the bone tissues.

There is also a category of temporary fixation implants, made up of a cupule punctured in its center by an orifice, and designed to be put in place between two bones, such as a metatarsal and a phalanx, by means of a provisional fixation pin of the cupule. Compared to the prostheses formed from two implants described previously, the temporary fixation articular implants present the advantage of being able to be easily removed once the fibrous tissues are reconstituted, in particular by means of the absence of definitive anchoring of these implants in the bone tissues.

By means of the provisional fixation pin, these implants can also be centerd and positioned precisely in the articular interface.

In spite of all these advantages, these implants nevertheless suffer from non-negligible disadvantages, connected in particular with the use of the temporary fixation pin.

In the first place, such implants require, for their emplacement, the making of an incision sufficiently wide to make possible correct centering of the pin. In the case of a metatarsal-phalangeal joint of the foot, the pin is thus generally introduced in the first place into the medullary canal of the successive phalanges, being directed towards the distal part of the toe, and in the second place into the medullary canal of the metatarsal by a to-and-fro technique. All these manipulations require the making of an incision that is relatively large and often much greater than the size of the implant. Now, it is desirable, as much from the aesthetic point of view as from the point of view of risks of infection and post-operatory pain, to reduce the size of the incisions that are made.

Furthermore, the emplacement of the implants of prior art has the disadvantage of quite frequently leading to damage of the articular capsule, in particular at the time of emplacement of the fixation pin by the to-and-fro technique.

Finally, the presence of the pin generally requires a new surgical intervention for the purpose of removing it, which complicates the consequences of the operation even more.

The implants of prior art therefore require, for their emplacement, a relatively unwieldy intervention, that may lead to several non-negligible complications, and the consequences of which are sometimes poorly tolerated by the patient.

Moreover, the implants of prior art generally retain, in spite of the presence of the fixation pin, certain mobility within the articular interface. In particular, implants held in position by means of a central fixation pin retain the capability of turning on themselves around the longitudinal axis of the pin. Now, this mobility of the implant may not only lead to a sensation of discomfort for the patient, but may also slow down the regeneration of bone tissue and cartilage. In addition, if the implant is poorly positioned, an abnormal erosion of the adjacent articular surfaces may occur. This phenomenon is observed in particular in the metatarsal-phalangeal joints of the foot on account of the significant constraints that are exerted there.

### SUMMARY

In response to these and other shortcomings of the prior art, an articular interposition implant is disclosed. The objects assigned to the invention consequently aim at proposing a novel articular interposition implant that does not present the disadvantages enumerated in the preceding and whose emplacement and positioning within the articular interface are particularly simple and rapid.

Another object of the invention aims at proposing a novel articular interposition implant which requires only a small incision for its emplacement.

Another object of the invention aims at proposing a novel articular interposition implant whose centering and positioning in the articular interface are facilitated.

Another object of the invention aims at proposing a novel articular interposition implant which would be particularly stable within the articular surface.

Another object of the invention aims at proposing a novel articular interposition implant whose probability of migration outside of its area of effectiveness within the articular interface is particularly low.

Another object of the invention aims at proposing a novel articular interposition implant requiring only a minimum of steps for its emplacement.

Another object of the invention aims at proposing a novel articular interposition implant which would be particularly simple to manufacture.

Another object of the invention aims at proposing a novel articular interposition implant facilitating the regeneration of bone tissue.

Another object of the invention aims at proposing a novel articular interposition implant adapted to the morphology of the metatarsal-phalangeal joint.

The objects assigned to the invention are attained by means of an articular interposition implant designed to be put in place between the articular surfaces (2A, 3A) of at least two bones (2, 3) separated by an articular interface 4 for the purpose of achieving arthroplasty of a joint 5, said implant 1 comprising immobilization means 6 shaped in such a way that when the latter is arranged in the articular interface 4, the immobilization means 6 are supportive of at least one of the articular surfaces (2A, 3A), characterised in that said implant 1 extends along a longitudinal axis (X-X′) and in that said immobilization means 6 are shaped so as to prevent rotation of said implant 1 on itself with respect to said longitudinal axis (X-X′).

Other systems, methods, features, and advantages of the invention will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1 depicts an articular interposition implant according to the invention put in place between a phalanx and a metatarsal.

FIG. 2 depicts, in a profile view, the articular interposition implant according to the invention.

FIG. 3 depicts, in a perspective view, the articular interposition implant according to the invention.

FIG. 4 depicts, in a top view, a variant of realisation of the articular interposition implant according to the invention.

FIG. 5 depicts, in a bottom view, the articular interposition implant according to the invention.

FIG. 6 and FIG. 7 depict, in a side view, two embodiments of the articular interposition implant according to the invention.

FIG. 8 and FIG. 9 depict, in a top view, two variants of realisation of the articular interposition implant according to the invention.

### DETAILED DESCRIPTION

Various aspects of the articular interposition implant, having been summarized above, reference will now be made in detail to the description of the representative assembly illustrated in the drawings. While the articular interposition implant will be described in connection with these drawings, there is no intent to limit it to the embodiment or embodiments disclosed therein.

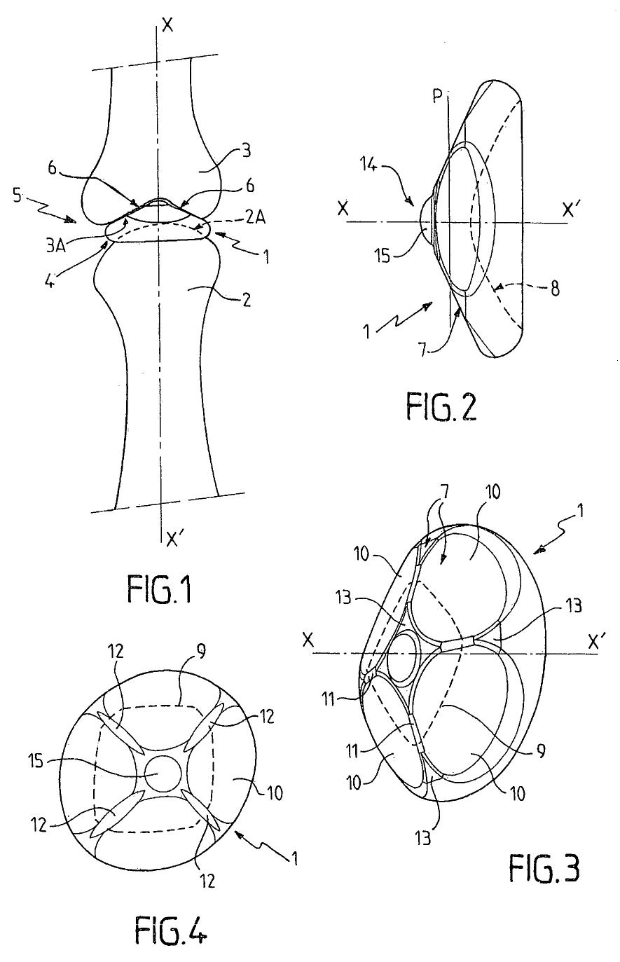

FIG. 1 depicts a joint 5 formed by two bones, for example a first bone 2 and a second bone 3, and an articular interposition implant 1 arranged at the interface between the two bones 2, 3. The first bone 2 may thus, for example, be formed by a metatarsal, the second bone 3 thus being, for example, formed by a phalanx. In this case, implant 1 to best advantage constitutes a metatarsal-phalangeal implant. However, joint 5 could quite obviously comprise a third bone, located, for example, between the first bone 2 and the second bone 3, and this without leaving the framework of the invention. Articular interposition implant 1 according to the invention is thus designed to be put in place between the articular surfaces 2A, 3A of bones 2, 3.

More precisely, articular interposition implant 1 is adapted to be introduced within articular interface 4 separating bones 2, 3 in order to achieve arthroplasty of joint 5. Arthroplasty thus consists in intervening surgically in a joint for the purpose of restoring its mobility. Such an operation is recommended in particular in the case in which the patient is suffering from a chronic degenerative disorder of the joints, such as arthrosis.

For the purpose of the invention, the term “implant” refers to a part that is preferably single and made of one piece, designed to be implanted within the joint for the purpose of improving its mobility, and which is thus differentiated from the prostheses, and in particular from the articulated prostheses made up of two parts, for example a metatarsal implant and a phalangeal implant set up mobile with respect to each other and fixed, respectively, at the end of the bones of the joint.

The expression “articular interposition implant” thus refers to a hinge-type implant, located at the interface between two bones in such a way as to restore their relative mobility.

In order to enable and also to facilitate bone reconstruction, it is often necessary to essentially immobilise implant 1, at least temporarily, within joint 5. To this end, and according to an essential characteristic of the invention, implant 1 according to the invention comprises immobilization means 6, shaped in such a way that when implant 1 is arranged in the articular interface 4, the immobilization means 6 are supportive of at least one of the articular surfaces 2A, 3A. Immobilization means 6 thus ensure, by friction and adherence to the articular surface 3A, automatic immobilization of implant 1 with respect to said articular surface 3A, and this without resorting to an anchoring pin or to any other temporary or permanent fixation component.

This spontaneous immobilization contributes to the progressive embedding of the implant by facilitating tissue regeneration which leads to the sealing of the latter against the articular surface 3A.

The surgical intervention may comprise a step for preparation of the articular surfaces, in particular of surface 3A, indeed for the fitting in of a housing, in particular in bone 3, to improve the seating of immobilization means 6 and consequently the stability of the implant. However, in a particularly advantageous way, immobilization means 6 are naturally supportive of articular surface 3A, without it being necessary to resect said articular surface, for example by arranging plane cut-outs in it. The articular surfaces 2A, 3A of bones 2, 3 being essentially curved, articular interposition implant 1 according to the invention to best advantage comprises at least first support surface 7, and preferentially a first and a second support surface 7, 8 as a whole curved and preferably approximately parallel with respect to each other. In an even more preferential way, the first and second support surfaces 7, 8 present essentially the same curvature. The first and second support surfaces 7, 8 thus to best advantage form a contact interface with the corresponding articular surfaces 2A, 3A.

The expression “curved on the whole” refers to the fact that the support surfaces 7, 8, although capable of comprising, locally, one or more portions of plane surfaces, present a general appearance that is curved, at least in pieces.

To best advantage, the first support surface 7 is thus convex on the whole, the second support surface 8 being concave on the whole. The articular interposition implant 1 thus presents the general shape of a cupule or half-shell, conferring on implant 1 an anatomical form that makes it possible for it to adapt itself to the natural morphology of the joint, and in particular to that of the metatarsal-phalangeal joint. Thanks to the anatomical form of implant 1, it is then unnecessary to resect articular surfaces 2A, 3A or the ends of bones 2, 3.

To best advantage, immobilization means 6 are of one piece with implant 1 and even more preferentially structurally integrated with implant 1, i.e. they are neither joined nor dissociated from said implant 1. This characteristic in particular makes it possible to automatically immobilise implant 1 within articular interface 4, at the time of its introduction, and therefore in a minimum of steps.

To best advantage, implant 1 extends, longitudinally, i.e. in a direction approximately perpendicular to the radial extension plane of the implant, along a longitudinal axis X-X′ which, when the implant is in position within the articular interface 4, is essentially coincident with the axis of the medullary canals of the first and second bones 2, 3 and, for example, of the corresponding metatarsal and phalanx.

Means of immobilization 6 are thus to best advantage shaped to prevent rotation of implant 1 on itself with respect to longitudinal axis X-X′. This makes it possible in particular to reduce the possibilities of mobility and play of the implant 1 within joint 5, capable not only of leading to discomfort for the patient, but also of leading to progressive wear of articular surfaces 2A, 3A. Rotation immobilization of implant 1 thus to best advantage makes it possible to promote regeneration of surrounding bone tissue.

According to a particularly advantageous characteristic of the invention, immobilization means 6 are arranged on first support surface 7 in such a way that the latter does not present rotational symmetry with respect to longitudinal axis X-X′. Thus, immobilization means 6 are arranged in such a way that first support surface 7 presents, along at least one line 9 referred to as parallel (by analogy to the parallels of a spherical or hemispherical surface defined with respect to the equator) located at the intersection between said first support surface 7 and at least one plane P perpendicular to the longitudinal axis X-X′, a discontinuous curvature. As a matter of fact, contrary to the implants of prior art, implant 1 according to the invention does not present rotational symmetry over its first support surface 7.

On the other hand, the second support surface 8, preferably concave, of implant 1 is to best advantage essentially spherical (FIG. 5) and presents rotational symmetry along longitudinal axis X-X′. By means of its spherical shape, the second support surface 8 of implant 1 makes possible the mobility of the first bone 2, along a spherical-type kinematic linkage (or spherical joint linkage), at the interface between the second support surface 8 that is on the whole concave and the articular surface 2A that is on the whole convex, corresponding to the first bone 2. Preferentially, the second support surface 8 is preferentially smooth so as to promote even more the mobility of joint 5. To best advantage, the restoration of the smoothness of movement (without jerk or excessive friction) can facilitate regeneration of damaged tissues, in particular cartilaginous, at the articular surface 2A.

Immobilization means 6, arranged on the first support surface 7, thus make it possible to essentially immobilise the implant 1 with respect to the second bone 3, thus promoting bone regeneration. The second support surface 8, on the contrary, promotes the mobility of the first bone 2 with respect to implant 1 on the one hand and the second bone 3 on the other hand.

Means of immobilization 6 can, however, be arranged on the second support surface 8, on the whole concave, and this without leaving the framework of the invention. In this case, the first support surface 7 presents an essentially smooth surface state, in order to enable the mobility of the second bone 3, for example the phalanx, with respect to implant 1.

According to a preferential embodiment of the invention depicted in particular in FIG. 3, means of immobilization 6 comprise at least one essentially plane surface 10 situated on the first support surface 7. As depicted in FIG. 3, the plane surface 10 is substantially inclined with respect to longitudinal axis X-X′. The plane surface 10 thus breaks the rotational symmetry of implant 1.

Immobilization means 6 could, without leaving the framework of the invention, also comprise one or more surfaces of curvature inverted with respect to the overall curvature of the first support surface 7. These surfaces could thus, in the case of a first support surface 7 that is on the whole convex, be formed from hollow (or concave) surfaces.

To best advantage, the plane surface 10 presents asperities (not shown), and for example striae facilitating the hooking and the adherence of implant 1 to the articular surface 3A situated opposite the first support surface 7, and therefore its rotational immobilization relative to said articular surface 3A. The rough aspect of the first support surface 7 is also likely to promote bone regeneration of the second bone 3, i.e., in the case of a metatarsal-phalangeal joint, of the phalanx.

In an even more preferential way, and as depicted in FIG. 3, the immobilization means 6 comprise a plurality of plane surfaces 10 situated on the first support surface 7, and substantially inclined with respect to each other in such a way as to form a polyhedron with principal axis Y-Y′.

For the purpose of the invention, the “principal axis” Y-Y′ of the polyhedron extends between the apex S and the fictitious base B of the polyhedron (depicted by dotted lines in FIG. 7), and in an essentially perpendicular way to said base B.

To best advantage, each plane surface 10 of implant 1 is inclined by an angle a between 45° and 75°, and preferentially on the order of 60° with respect to principal axis Y-Y′ of the polyhedron.

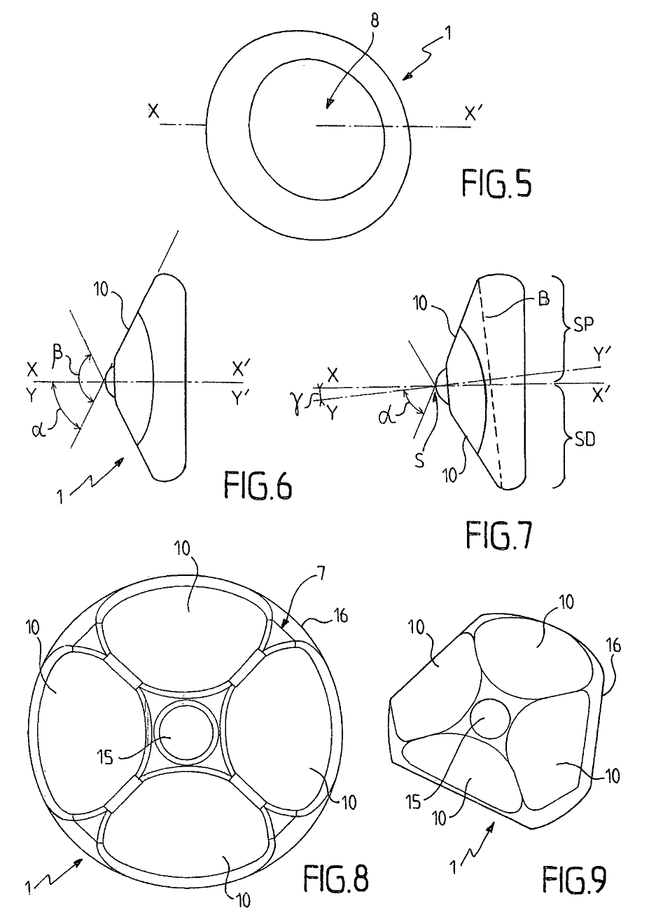

As depicted in FIG. 6, angle β at the apex S of the polyhedron has a value between 90° and 150°, preferentially on the order of 120°.

The plane surfaces 10 are preferentially juxtaposed in such a way as to present, two by two, at least one common side 11. According to a variant of realisation depicted in FIG. 4, implant 1 comprises, between two successive plane surfaces 10, a connecting flange 12. It is, however, quite obviously conceivable to construct an implant 1 in which the plane surfaces 10 are connected to each other without excessive thickness, and therefore without a connecting flange.

According to an even more preferential variant of realisation of the invention, the immobilization means 6 are formed by four juxtaposed plane surfaces 10 arranged in the shape of a pyramid (FIG. 3).

This particular form of implant 1 makes it possible for it to penetrate substantially and effectively within the hollow formed by the concave articular surface 3A.

To best advantage, the first support surface 7 of implant 1 is formed from a combination of plane surfaces 10 and curved surfaces 13 oriented in space with respect to each other in such a way as to confer on the first support surface 7 a shape that is on the whole convex. The first support surface 7 thus presents a plurality of discontinuities in its curvature, in particular in a plane P that is essentially median and perpendicular to the longitudinal axis X-X′ of implant 1.

According to a variant of realisation depicted in FIG. 6, principal axis Y-Y′ of the polyhedron is essentially coincident with longitudinal axis X-X′ of implant 1, which also corresponds to the axis of rotational symmetry of the concave second support surface 8.

According to another variant of realisation depicted in FIG. 7, the principal axis Y-Y′ of the polyhedron is essentially inclined by an angle γ with respect to longitudinal axis X-X′. Preferentially, principal axis Y-Y′ of the polyhedron is inclined by an angle γ between 5° and 25° with respect to longitudinal axis X-X′.

The inclination of the polyhedron thus makes it possible to reproduce the morphology of certain joints, in particular metatarsal-phalangeal joints, for which the bones, i.e. the metatarsal and the phalanx, are not necessarily aligned but slightly inclined with respect to each other. Thus, an implant 1 designed to be positioned between a phalanx and a metatarsal of the foot will present to best advantage a specific dimensioning.

The metatarsal-phalangeal implant 1 according to the invention thus preferably presents an average thickness on the order of 4 mm but may also comprise variations of thickness in such a way as to reproduce the dorsiflexion of joint 5. Implant 1 may thus be broken down into several angular sections of different thicknesses. In the case in which implant 1 is dimensioned to be positioned between a phalanx and a metatarsal of the foot, it breaks down preferentially into a plantar section SP, situated on the sole of the foot when it is put in place within joint 5, and a dorsal section SD, situated on the dorsal part of the foot when implant 1 is put in place, plantar section SP presenting a thickness appreciably greater than that of dorsal section SD, in such a way as to reproduce the natural inclination of the metatarsal and the phalanx.

In a particularly advantageous way, the thickness ratio between the plantar section and the dorsal section is between 1.5 and 2.5.

Implant 1 according to the invention is in addition to best advantage dimensioned in such a way that it can be slid with a slight clearance between articular surfaces 2A, 3A in such a way that once positioned in articular interface 4, implant 1 is not situated at a distance from articular surfaces 2A, 3A but in contact with the latter.

The diameter of implant 1 is also adjusted to the diameter of the bones 2, 3 forming the joint, in such a way that, in the worst case, it protrudes only very slightly outside joint 5.

According to another particularly advantageous characteristic of implant 1 according to the invention, and which moreover constitutes an invention in its own right, implant 1 comprises self-centering means 14 shaped to be supportive of at least one of the articular surfaces 2A, 3A, thus ensuring automatic diametral centering of implant 1 with respect to bones 2, 3.

The self-centering means are thus to best advantage shaped to make the central longitudinal axis X-X′ of implant 1 approximately coincide with the axes of the medullary canals of the first and second bones 2, 3.

To best advantage, the auto-centering means 14 are preferentially arranged on the first support surface 7, on the whole convex, of implant 1 in such a way as to be supportive of the articular surface 3A, essentially concave, of the second bone 3.

More precisely, the self-centering means 14 are to best advantage shaped to be supportive in an essentially point-to-point way of articular surface 3A situated opposite, and to be positioned naturally, in a position of stable equilibrium, in the deepest part of articular surface 3A.

To this end, self-centering means 14 are preferentially provided with at least one protuberance 15, such as a nipple, protruding essentially in the center of the implant from the first support surface 7 which is on the whole convex. Protuberance 15 is thus approximately aligned with the central longitudinal axis X-X′, and extends essentially in the same direction as the latter.

Contrary to the implants of prior art, implant 1 according to the invention therefore lacks a central orifice and presents itself to best advantage in the form of an implant that is essentially solid and of one-piece construction.

According to a preferential variant of realisation of the invention depicted in FIG. 8, protuberance 15 and plane surfaces 10 are to best advantage arranged in staggered fashion, protuberance 15 being situated approximately at the center of implant 1, and the plane surfaces 10 on the periphery of implant 1.

As depicted in FIG. 8 and FIG. 9, the first and second support surfaces 7, 8 join along a boundary line 16 which, according to the case, may be circular (FIG. 8) or polygonal (FIG. 9).

Preferentially, articular interposition implant 1 is constructed, at least partially, of pyrocarbon so as to promote regeneration of bone tissue. Pyrocarbon thus makes it possible, by means of its mechanical properties, which are essentially identical to those of bone tissue, to obtain low friction couples between implant 1 and bones 2, 3 forming the joint, thus limiting the phenomenon of bone lysis.

However, it is conceivable to use, for construction of the implant, another bio-compatible material whose mechanical properties are adapted to the application described, in particular ceramics or metallic materials.

The method for placement of implant 1 according to the invention will now be described with reference to FIG. 1 through FIG. 9.

The surgical placement method for implant 1 according to the invention comprises in the first place an incision step, during which an incision, of dimensions at least equal to, and scarcely greater than those of implant 1, is made in joint 5 in such a way as to enable introduction of implant 1. According to the degree of degradation of joint 5, the surgeon may or may not eliminate the irregularities from articular surfaces 2A, 3A opposite, so as to confer on them a shape that is appreciably smoother and more rounded.

The surgical method for arthroplasty according to the invention comprises next a step (a) for immobilization of articular interposition implant 1 between articular surfaces 2A, 3A of bones 2, 3, during which implant 1 is directly positioned in the articular interface 4 in such a way that it is supportive of, and is immobilised through its own immobilization means 6, at least one of the articular surfaces 2A, 3A.

Thus, the implant is immobilised without resorting to any accessory, or fixation unit, or any added immobilization means, such as, for example, an immobilization or fixation pin.

The method according to the invention comprises, in addition, to best advantage, a step (b) for self-centering of implant 1 during which the implant 1 is directly positioned in the articular interface 4 in such a way that it is supportive, through is own self-centering means 14, of at least one of articular surfaces 2A, 3A, so as to center itself spontaneously.

Thus, centering of the implant is attained intuitively and immediately, without having to resort to any means for positioning or to any accessory centering device, such as, for example, a centering pin.

In a particularly advantageous way, steps (a) for immobilization and (b) for self-centering of implant 1 are carried out simultaneously.

Moreover, during self-centering step (b), the self-centering means 14 of implant 1 are positioned to be supportive of the essentially concave articular surface 3A of one of bones 2, 3, in such a way that said self-centering means 14 are positioned naturally in the deepest part of articular surface 3A. In this way, protuberance 15 and plane surfaces 10 are essentially simultaneously supportive of articular surface 3A, thus simultaneously ensuring, on the one hand, the diametral centering of implant 1 within articular interface 4 and, on the other hand, its rotational immobilization, by means in particular of the adherence of plane surfaces 10 to articular surface 3A.

To best advantage, after the steps for (a) immobilization and (b) self-centering, implant 1 is allowed to rest in articular interface 4, without fixing it in position.

The surgical method according to the invention therefore makes it possible, by means of a single operative action, to center and immobilise implant 1 within articular interface 4, thus doing away with the standard additional steps of centering and fixation of the implant by means of a pin.

The emplacement of articular interposition implant 1 according to the invention does not to best advantage require any preliminary preparation of the articular surfaces, and in particular does not require any milling operation, thanks in particular to the first and second on the whole curved support surfaces of implant 1.

Another advantage of implant 1 according to the invention is that it makes it possible, not only by means of the nature of the materials of which it is constituted, but also by means of its anatomical shape and the immobilization means 6 structurally integrated in implant 1, to limit the phenomenon of bone lysis.

Another advantage of implant 1 according to the invention is that its placement is rapid and is carried out in a minimum number of steps, which makes it possible to limit the risk of complications and operatory errors.

Now, therefore, at least the following is claimed:

1. Articular interposition implant to be placed between articular surfaces (2A, 3A) of at least two bones (2, 3) separated by an articular interface (4) to achieve arthroplasty of a joint (5), said implant (1) comprising immobilization means (6) shaped so that when the immobilization means are arranged in the articular interface (4), the immobilization means (6) are supportive of at least one of the articular surfaces (2A, 3A), wherein said implant (1) extends along a longitudinal axis (X-X′) and wherein said immobilization means (6) are shaped to prevent rotation of said implant (1) on itself with respect to said longitudinal axis (X-X′).

2. Implant as claimed in claim 1, wherein the immobilization means (6) are of one piece with the implant (1).

3. Implant as claimed in claim 1, further comprising at least a first support surface (7), essentially curved so as to form a contact interface with one of the articular surfaces (2A, 3A), the immobilization means (6) being arranged on said first support surface (7) such that an intersection between said first support surface (7) and at least one plane (P) perpendicular to the longitudinal axis (X-X′) forms a line of discontinuous curvature.

4. Implant as claimed in claim 3, wherein the immobilization means (6) further comprises at least one essentially plane surface (10) situated on said first support surface (7).

5. Implant as claimed in claim 4, wherein said plane surface (10) is substantially inclined with respect to the longitudinal axis (X-X′).

6. Implant as claimed in claim 4, wherein said plane surface (10) presents asperities, for example striae.

7. Implant as claimed in claim 3, further comprising a first and a second support surface (7, 8), essentially curved and essentially parallel with respect to each other.

8. Implant as claimed in claim 7, wherein the first support surface (7) is essentially convex and the second support surface (8) is essentially concave.

9. Implant as claimed in claim 4, wherein the immobilization means (6) further comprises a plurality of plane surfaces (10), substantially inclined with respect to each other, to form a polyhedron.

10. Implant as claimed in claim 9, wherein the angle (p) at the apex (S) of the polyhedron has a value between 90° and 150°.

11. Implant as claimed in claim 9, wherein said plane surfaces (10) are juxtaposed to present, two by two, at least one common side (11).

12. Implant as claimed in claim 11, further comprising a connecting flange (12) between two successive plane surfaces (10).

13. Implant as claimed in claim 9, wherein the immobilization means (6) are formed by four juxtaposed plane surfaces (10) arranged in a pyramid shape.

14. Implant as claimed in claim 1, further comprising self-centering means (14), shaped to be supportive of at least one of the articular surfaces (2A, 3A), thus ensuring automatic centering of implant (1) with respect to bones (2, 3).

15. Implant as claimed in claim 3, further comprising self-centering means (14), shaped to be supportive of at least one of the articular surfaces (2A, 3A), thus ensuring automatic centering of implant (1) with respect to bones (2, 3), wherein the self-centering means (14) are arranged on the first support surface (7).

16. Implant as claimed in claim 14, wherein the self-centering means (14) are provided with at least one protuberance (15), protruding essentially from a center of the implant (1).

17. Implant as claimed in claim 13, further comprising self-centering means (14), shaped to be supportive of at least one of the articular surfaces (2A, 3A), thus ensuring automatic centering of implant (1) with respect to bones (2, 3), wherein the self-centering means (14) are provided with at least one protuberance (15), protruding essentially from a center of the implant (1), wherein the protuberance (15) and the plane surfaces (10) are arranged in a staggered fashion, the protuberance (15) being situated essentially in the center of the implant (1), with the plane surfaces (10) on a periphery of the implant (1).

18. Implant as claimed in claim 9, wherein a principal axis (Y-Y′) of the polyhedron is approximately coincident with the longitudinal axis (X-X′).

19. Implant as claimed in claim 9, wherein a principal axis (Y-Y′) of the polyhedron is substantially inclined with respect to longitudinal axis (X-X′).

20. Implant as claimed in claim 19, wherein the principal axis (Y-Y′) of the polyhedron is inclined by an angle (α) between 5° and 25° with respect to the longitudinal axis (X-X′).

21. Implant as claimed in claim 3, wherein the first support surface (7) of implant (1) is formed by a combination of plane surfaces (10) and curved surfaces (13), oriented in space to confer a shape that is essentially convex on said first support surface (7).

22. Implant as claimed in claim 1, wherein the implant (1) is made at least partially of pyrocarbon.

23. Implant as claimed in claim 1, wherein the implant (1) comprises a plantar section (SP) and a dorsal section (SD), the plantar section (SP) presenting a thickness appreciably greater than that of the dorsal section (SD).

24. Implant as claimed in claim 1, wherein the implant (1) is essentially solid constitutes an implant (1) in one piece.

25. Implant as claimed in claim 1, wherein the implant (1) is dimensioned to be positioned between a phalanx and a metatarsal of the foot or hand, and thus constitutes a metatarsal-phalangeal interposition implant (1).

26. Surgical method for arthroplasty by placement of an articular interposition implant between the articular surfaces (2A, 3A) of at least two bones (2, 3) separated by an articular interface (4) said method comprising the steps of:
immobilizing the implant and positioning the implant in the articular interface (4) in such a way that it is supportive, and is immobilised through its own immobilization means (6), of at least one of the articular surfaces (2A, 3A);
self-centering the implant by positioning the implant in the articular interface (4) in such a way that it is supportive, through its own auto-centering means (14), of at least one of the articular surfaces (2A, 3A), so as to center itself.

27. The method of claim 26, wherein during the self-centering step, the self-centering means (14) of the implant are positioned to be supportive of the essentially concave articular surface (3A) of one of bones (2, 3), in such that said self-centering means (14) are positioned naturally in a deepest part of the articular surface (3A).

28. The method of claim 27, wherein the plane surfaces (10) are essentially simultaneously supportive of the articular surface (3A), thus simultaneously ensuring diametral centering of the implant within the articular interface (4) and its rotational immobilization, by means of the adherence of the plane surfaces (10) to the articular surface (3A).

29. The method of claim 26, wherein the immobilising step and the self-centering step of the implant (1) are carried out simultaneously.

30. The method of claim 26, wherein after the immobilizing step and self-centering step, implant (1) is allowed to rest in articular interface (4), without fixing said implant in place.

31. The method of claim 26, wherein emplacement of the implant does not require preparation of the articular surfaces.

32. Implant as claimed in claim 15, wherein the self-centering means (14) are provided with at least one protuberance (15), protruding essentially from a center of the implant (1).

33. Implant as claimed in claim 13, further comprising at least a first support surface (7), essentially curved so as to form a contact interface with one of the articular surfaces (2A, 3A), the immobilization means (6) being arranged on said first support surface (7) such that an intersection between said first support surface (7) and at least one plane (P) perpendicular to the longitudinal axis (X-X′) forms a line of discontinuous curvature, further comprising self-centering means (14), shaped to be supportive of at least one of the articular surfaces (2A, 3A), thus ensuring automatic centering of implant (1) with respect to bones (2, 3), wherein the self-centering means (14) are arranged on the first support surface (7), wherein the self-centering means (14) are provided with at least one protuberance (15), protruding essentially from a center of the implant (1), wherein the protuberance (15) and the plane surfaces (10) are arranged in a staggered fashion, the protuberance (15) being situated essentially in the center of the implant (1), with the plane surfaces (10) on a periphery of the implant (1).
